(19) United States
(12) Patent Application Publication
(10) Pub. No.: US 2024/0050667 A1
(43) Pub. Date: Feb. 15, 2024

(54) AEROSOLIZATION WITHIN RESPIRATORY SYSTEM

(71) Applicants: Regents of the University of Minnesota, Minneapolis, MN (US); Abbe Vision Inc., Eden Prairie, MN (US)

(72) Inventors: Amir Ahmad Naqwi, Eden Prairie, MN (US); Kari Diane Roberts, St. Paul, MN (US)

(21) Appl. No.: 18/255,982

(22) PCT Filed: Dec. 6, 2021
(86) PCT No.: PCT/US2021/061957

§ 371 Date: Jun. 5, 2023

Related U.S. Application Data
(60) Provisional application No. 63/122,041, filed on Dec. 7, 2020.

(51) Int. Cl. A61M 11/02; A61M 16/04; A61M 16/08

(52) U.S. Cl. CPC A61M 11/02; A61M 16/0402; A61M 16/0816; A61M 16/0434; A61M 2202/0488

(57) ABSTRACT

A system for administering an aerosolized liquid to a respiratory system of a patient includes a patient interface and an aerosolization device. The patient interface includes a laryngeal mask airway comprising a tracheal tube and a laryngeal cuff located at a distal end of the tracheal tube. The aerosolization device includes a liquid delivery device configured to deliver a liquid, a gas flow generator configured to generate a gas flow, and a sprayer. The sprayer includes a catheter configured to extend into the tracheal tube. The catheter includes a first lumen configured to receive the gas flow at a proximal end, a second lumen configured to receive a flow of the liquid at the proximal end, and a spray tip at a distal end configured to aerosolize the flow of the liquid using the gas flow. The first lumen extends through the second lumen.

### CROSS-REFERENCE TO RELATED APPLICATION

The present application is based on and claims the benefit of U.S. provisional patent application Ser. No. 63/122,041, filed Dec. 7, 2020, the content of which is hereby incorporated by reference in its entirety.

### FIELD

Embodiments of the present disclosure relate to systems and methods for delivering aerosolized liquids, such as therapeutics and other agents, to the respiratory system of a patient.

### BACKGROUND

Respiratory conditions of patients are often treated through the delivery of a therapeutic into the respiratory system. For example, exogenous lung surfactant is used to treat preterm infants with respiratory distress syndrome. Such treatments conventionally administer the surfactant via a bolus through an endotracheal tube, which requires highly invasive intubation (placement of an endotracheal tube) and mechanical ventilation, which can have injurious effects on the infant. Bolus administration is also associated with oxygen desaturation and bradycardia as a result of the surfactant filling the airways prior to absorption into the lung alveoli.

Devices such as a metered dose inhaler (MDI) and a dry powder inhaler (DPI) are satisfactory for patients who have the ability to coordinate the device operation with inhalation and breath-holding to ensure effective drug delivery. Many patients, including neonates, young pediatric patients and patients requiring assisted breathing, do not have this ability. Therefore, efforts have been made to develop a catheter-like aerosolizer that can be used for aerosolized delivery to such patients.

U.S. Pat. No. 5,803,078 discloses an endotracheal assembly with a multi-lumen catheter, whose distal tip is positioned in the trachea near the carina of the patient. Separate lumens are used to deliver gas and liquid that interact at the distal tip to generate aerosols. U.S. Pat. No. 5,964,223 discloses a method using an aerosolization mechanism that is similar to that disclosed in the '078 patent. As generally illustrated in FIG. 14A, the method utilizes an endotracheal tube (ETT) 300 that is inserted into a patient's trachea 132 while the patient is under anesthesia, and a ventilator is used to support breathing. The catheter used to discharge an aerosolized liquid 302 is inserted while the ETT remains in place, as illustrated in FIG. 14A. As a result, the disclosed technique involves a substantially invasive procedure.

U.S. Pat. No. 10,456,538 discloses a nebulizing (or atomizing) catheter 304, whose distal tip is placed in the retro-pharyngeal region 306 of a spontaneously breathing neonate, as generally illustrated in FIG. 14B. The disclosed apparatus and method eliminate the use of an ETT, but are incapable of confining the application of the aerosolized liquid 302 to the respiratory system of the patient.

There is a continued demand for improved methods of delivering therapeutics into the respiratory system of patients.

### SUMMARY

Embodiments of the present disclosure relate to systems and methods for delivering aerosolized liquids, such as therapeutics and other agents, to the respiratory system of a patient. One embodiment of the system includes a patient interface and an aerosolization device. The patient interface includes a laryngeal mask airway comprising a tracheal tube and a laryngeal cuff located at a distal end of the tracheal tube. The aerosolization device includes a liquid delivery device configured to deliver a liquid, a gas flow generator configured to generate a gas flow, and a sprayer. The sprayer includes a catheter configured to extend into the tracheal tube. The catheter has proximal and distal ends and includes a first lumen configured to receive the gas flow at the proximal end, a second lumen configured to receive a flow of the liquid at the proximal end, and a spray tip at the distal end configured to aerosolize the flow of the liquid using the gas flow. The first lumen extends through the second lumen.

A method for administering an aerosolized liquid to the respiratory system of a patient uses a system that includes a patient interface including a laryngeal mask airway comprising a tracheal tube and a laryngeal cuff located at a distal end of the tracheal tube, and an aerosolization device. The aerosolization device includes a liquid delivery device, a gas flow generator, and a sprayer having a catheter including first and second lumens extending from a proximal end to a distal end, and a spray tip at the distal end. The first lumen extends through the second lumen. In the method, the distal end of the tracheal tube is inserted into the throat of the patient, and the laryngeal cuff is positioned in the pharynx of the throat. The distal end of the catheter is inserted into the tracheal tube. A gas flow generated by the gas flow generator is delivered through the first lumen from the proximal end of the catheter to the spray tip. A flow of a liquid discharged from the liquid delivery device is delivered through the second lumen from the proximal end of the catheter to the spray tip. The liquid is aerosolized using the gas flow at the spray tip. The aerosolized liquid to the respiratory system of the patient through the tracheal tube.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the Background.

### BRIEF DESCRIPTION OF THE DRAWINGS

FIGS. 1 and 2 are simplified diagrams of a system for administering an aerosolized liquid to a respiratory system of a patient, in accordance with embodiments of the present disclosure.

FIG. 3 is a simplified diagram illustrating an example of the system, in accordance with embodiments of the present disclosure.

FIG. 4 is a simplified diagram of an example of a controller, in accordance with embodiments of the present disclosure.

FIG. 5 is a simplified diagram of an example of the aerosolization device, in accordance with embodiments of the present disclosure.

FIG. 6 is a simplified diagram of a sprayer, in accordance with embodiments of the present disclosure.

FIG. 7 is a simplified side cross-sectional view of a catheter of the sprayer of FIG. 6, in accordance with embodiments of the present disclosure.

FIG. 8 is a simplified cross-sectional view of the catheter of the sprayer of FIG. 6, in accordance with embodiments of the present disclosure.

FIG. 9 is a simplified cross-sectional view of an example of a spray tip of a sprayer, in accordance with embodiments of the present disclosure.

FIGS. 10A and 10B are simplified diagrams illustrating net forces on an example of a central lumen during operation, in accordance with embodiments of the present disclosure.

FIG. 11 is a simplified side cross-sectional view of an example of a spray tip of the sprayer of FIG. 6, in accordance with embodiments of the present disclosure.

FIG. 12 is a simplified side cross-sectional view of an example of an adapter for coupling a gas flow and a liquid flow to lumens of a catheter of a sprayer, in accordance with embodiments of the present disclosure.

FIG. 13 is a flowchart illustrating a method for administering an aerosolized liquid to the respiratory system of a patient, in accordance with embodiments of the present disclosure.

FIGS. 14A and 14B are simplified diagrams of therapeutic delivery systems in operation with a patient, in accordance with the prior art.

### DETAILED DESCRIPTION OF ILLUSTRATIVE EMBODIMENTS

Embodiments of the present disclosure are described more fully hereinafter with reference to the accompanying drawings. Elements that are identified using the same or similar reference characters refer to the same or similar elements. The various embodiments of the present disclosure may, however, be embodied in many different forms and should not be construed as limited to the embodiments set forth herein. Rather, these embodiments are provided so that this disclosure will be thorough and complete, and will fully convey the scope of the present disclosure to those skilled in the art.

Specific details are given in the following description to provide a thorough understanding of the embodiments. However, it is understood by those of ordinary skill in the art that the embodiments may be practiced without these specific details. For example, circuits, systems, networks, processes, frames, supports, connectors, motors, processors, and other components may not be shown, or may be shown in block diagram form in order to not obscure the embodiments in unnecessary detail.

Embodiments of the present disclosure relate to improved techniques for delivering aerosolized liquids to the respiratory system of patients in a minimally invasive manner. Delivery of a therapeutic in an aerosol form facilitates more uniform dispersion of the therapeutic within the respiratory system of the patient over liquid bolus forms. Additionally, aerosolization of the therapeutic enables deeper penetration into the respiratory system.

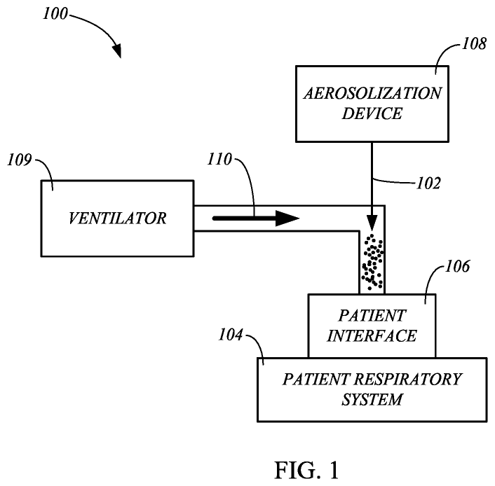

FIG. 1 is a simplified diagram of a system 100 for administering an aerosolized liquid 102 to a respiratory system 104 of a patient, in accordance with embodiments of the present disclosure. The liquid may take on any suitable form. In some embodiments, the liquid may comprise a therapeutic, such as an exogenous lung surfactant for use in treating preterm infants with respiratory distress syndrome. Other therapeutics or agents that may be aerosolized by the system 100 include, but are not limited to, corticosteroids, bronchodilators, antibiotics, and advanced therapeutics targeting specific cells in the respiratory system.

Embodiments of the system 100 may include a patient interface 106 and an aerosolization device 108. The system may include or utilize a ventilator 109 that generates a ventilator airflow 110 that assists the patient in breathing, or breathes for the patient. The interface 106 may be a conventional device used to connect the ventilator airflow 110 to the patient's respiratory system 104.

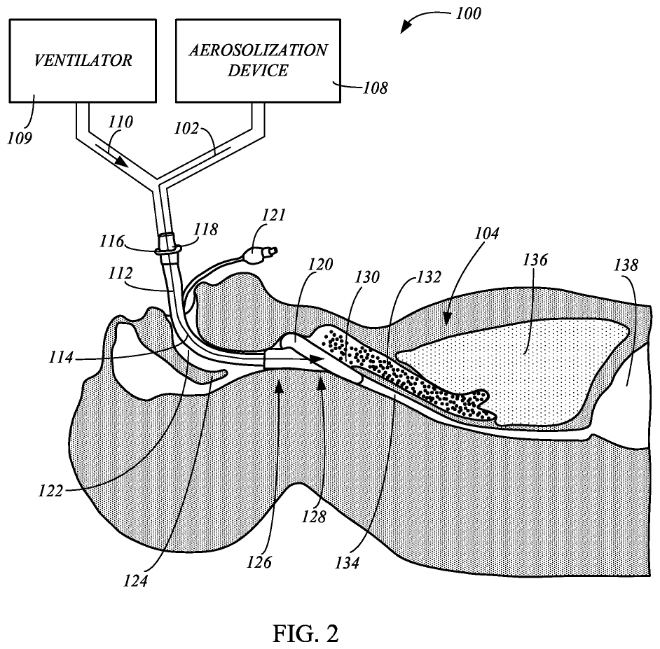

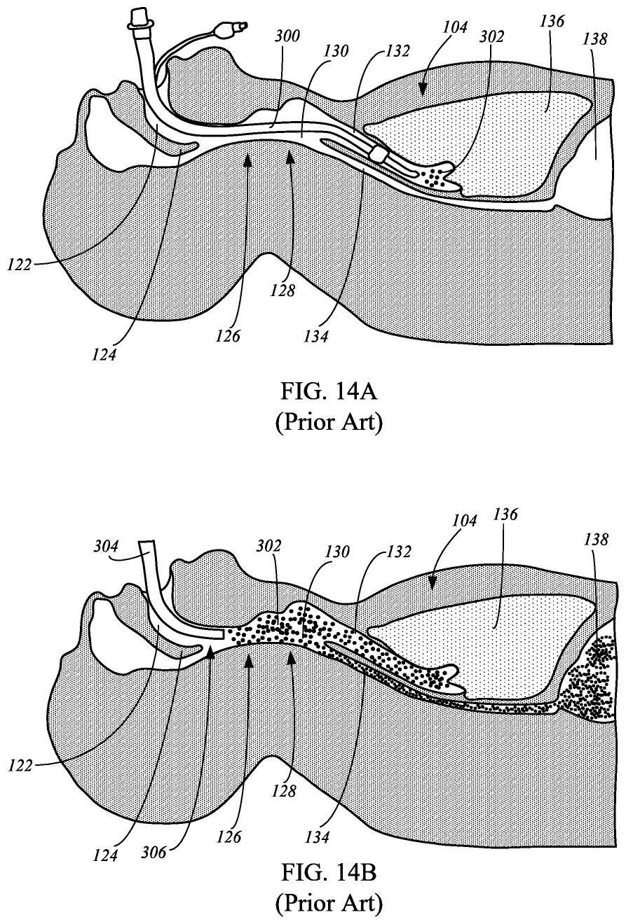

In one embodiment, the interface 106 comprises a laryngeal mask airway (LMA) 112, such as shown in FIG. 2, which is a simplified diagram of the system 100 in use with a patient, in accordance with embodiments of the present disclosure. The use of the LMA 112 provides benefits over the intubation technique shown in FIG. 14A, such as faster and easier placement, and less physiologic changes compared to intubation, for example. The aerosolization device 108 operates to aerosolize a liquid and discharge the aerosolized liquid 102 to a desired location within the respiratory system 104 of the patient through the interface 106, such as the entrance to the pharynx, as illustrated in FIG. 2.

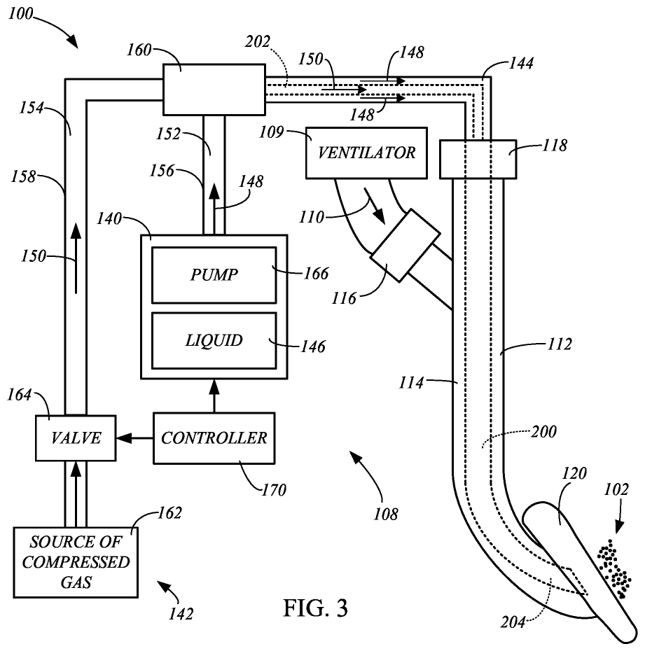

FIG. 3 is a simplified diagram of an example of the system 100, in accordance with embodiments of the present disclosure. The LMA 112 may have a conventional form, such as that disclosed in U.S. Pat. Nos. 11,135,385 and 6,895,966. In one example, the LMA 112 includes a tracheal or airway tube 114, a connector 116 for connecting the ventilator to the tracheal tube 114, a port 118 (FIG. 3) through which one or more components of the aerosolization device 108 may be inserted into the tracheal tube 114, and a laryngeal cuff 120 at a distal end of the tube 114, which may be inflated and deflated using a suitable inflation mechanism 121 (FIG. 2).

The LMA 112 may be inserted into the throat 122 of the patient, the upper surface of which is bounded by the hard palate 124 and the soft palate 126, and placed in the pharynx 128 at the base of the hypopharynx 130 where the throat 122 divides into the trachea 132 and the esophagus 134, as shown in FIG. 2. With a lower portion of the cuff 120 placed at the base of the hypopharynx 130, the cuff 120 is inflated to lodge the LMA 112 in the pharynx 128. As a result, the aerosolized liquid 102 discharged by the aerosolization device 108 is directed to the lungs 136 of the patient using the LMA 112 without intubation, as with the prior art technique shown in FIG. 14A, and without dispensing the aerosolized liquid 102 to the stomach 138 of the patient through the esophagus 134, as with the prior art technique shown in FIG. 14B.

The aerosolization device 108 generally includes a liquid delivery device 140, as gas flow generator 142, and a sprayer 144. The liquid delivery device 140 is configured to deliver a liquid 146, such as a flow of liquid 148, that is to be aerosolized and form the aerosolized liquid 102. The gas flow generator is configured to generate a gas flow 150 (e.g., air or oxygen) that is used to aerosolize the liquid flow 148.

The liquid flow 148 and the gas flow 150 may be provided to the sprayer 144 respectively through flow pathways 152 and 154, which may be formed by tubing 156 and 158. An adapter or connector 160 may be used to facilitate routing of the liquid flow 148 and the gas flow 150 to specific components of the sprayer 144, such as distinct lumens of the sprayer 144, as described below in greater detail. The sprayer 144 operates to deliver the liquid flow 148 and the gas flow 150 to a desired location within the interface 106, such as the distal end of the trachea tube 114 or within the trachea 132 of the patient, where they are combined to form the aerosolized liquid 102, which may be combined with the ventilator airflow 110 for delivery to the patient's respiratory system 104 through the interface 106 (e.g., LMA 112).

One example of the gas flow generator 142 comprises a source of compressed gas 162, and one or more valves (e.g., solenoid valves), such as valve 164 shown in FIG. 3, that are used to generate one or more gas flows, including the gas flow 150 through the flow pathway 154.

The liquid delivery device 140 operates to discharge the liquid flow 148 from a supply of the liquid 146 to the flow pathway 152. In some embodiments, the liquid delivery device 140 comprises a pump 166, such as a syringe, a syringe pump, a piston pump, a metering pump, a pneumatic pump, a gear pump, a rotary pump, and/or another suitable pump. The liquid delivery device 140 may meter the liquid flow 148 to a desired volumetric flow rate, such as 2 milliliters per minute, or another desired volumetric flow rate, using any suitable technique.

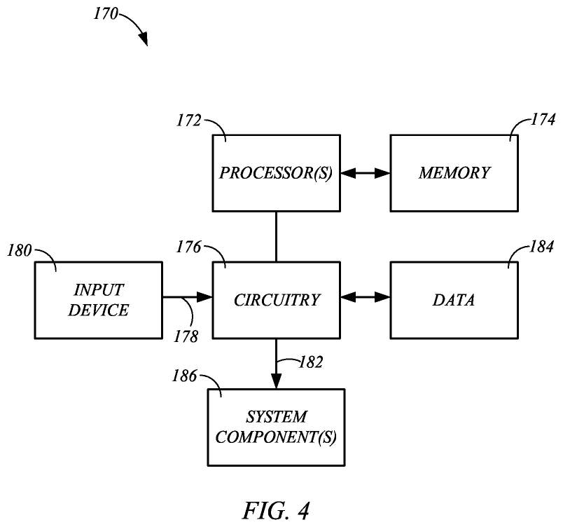

The system 100 may include a controller 170 for controlling valves, such as the valve 164, the pump 166 of the liquid delivery device 140, and/or other components of the system 100, as indicated in FIG. 3. FIG. 4 is a simplified diagram of an example of the controller 170, in accordance with embodiments of the present disclosure.

The controller 170 may include one or more processors 172 and memory 174. The one or more processors 172 are configured to perform various functions described herein in response to the execution of instructions contained in the memory 174. Examples of the functions that may be performed by the controller 170, such as in response to a user input or received data, include controlling the volumetric flow rate of the gas flow 150 and/or the liquid flow 148, setting operational parameters of components of the system 100, such as the device 108 and the ventilator 109, receiving and processing information and signals, such as information relating to the ventilator flow 110 (e.g., flow rate), and/or other functions.

The one or more processors 172 of the controller 170 may be components of one or more computer-based systems, and may include one or more control circuits, microprocessor-based engine control systems, and/or one or more programmable hardware components, such as a field programmable gate array (FPGA). The memory 174 represents local and/or remote memory or computer readable media. Such memory comprises any suitable patent subject matter eligible computer readable media that do not include transitory waves or signals such as, for example, hard disks, CD-ROMs, optical storage devices, and/or magnetic storage devices.

The controller 170 may include circuitry 176 for use by the one or more processors 172 to receive input signals 178 from one or more input devices 180 (e.g., switch), issue control signals 182 to system components 186 (e.g., valves), and or communicate data 184, such as in response to the execution of the instructions stored in the memory 174.

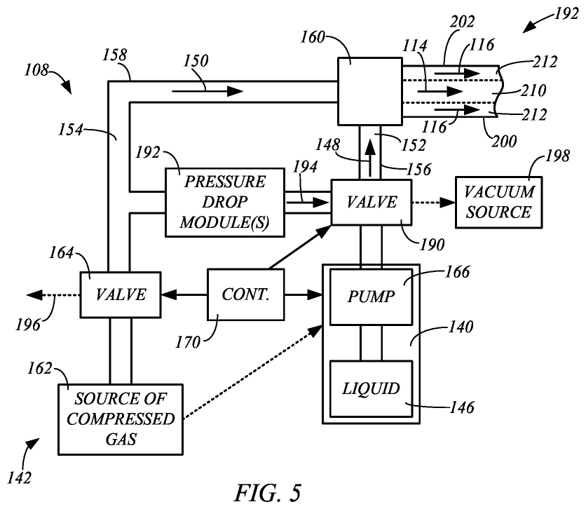

FIG. 5 is a simplified diagram of an example of the aerosolization device 108, in accordance with embodiments of the present disclosure. In this example, the liquid delivery device 140 makes use of the source of compressed gas 162 to deliver the liquid or liquid flow 148 to the sprayer 144. In one embodiment, the device 108 includes a valve 190 that is fluidically connected to the source of compressed gas 162, the pump 166 or the liquid 146 delivered by the pump 166, and the flow pathway 152 or tube 156 that is connected to the sprayer 144, such as the connector or adapter 160 of the sprayer 144. The valve 190 may be adjusted to one or more settings either manually or using the controller 170 to selectively connect the flow pathway 152 to the source of compressed gas 162 and the pump 166.

The connection of the valve 190 to the source of compressed gas 162 may be through the flow pathway 154 controlled by the valve 164, as shown in FIG. 5, or through a separate flow pathway. One or more conventional pressure drop modules 192 may be used to reduce the pressure at the valve 190, such that the gas flow to the valve is reduced from the gas flow 150 delivered to the sprayer 144.

In operation, the valve 190 is adjusted to a setting, in which the flow pathway 152 is fluidically connected to the pump 166, and the source of compressed gas 162 is fluidically disconnected from the flow pathway 152. The pump 166 is activated to drive the liquid 146 through the valve 190 and into the flow pathway 152. This activation of the pump 166 may involve the manual operation of a syringe containing the liquid 146, or through the control of the pump 166 by the controller 170, such that a desired volume of the liquid 146 is contained in the flow pathway 152.

The valve 190 is then adjusted to a setting, in which the source of compressed gas 162 is fluidically connected to the flow pathway 152, and the pump 166 is fluidically disconnected from the flow pathway 152. This causes a gas flow 194 from the source of compressed gas 162 to drive the liquid 146 within the flow pathway 152 to the sprayer 144, and through the sprayer 144 where it may be aerosolized by the gas flow 150 into the aerosolized liquid 102, as discussed below in greater detail.

When it is desirable to stop the discharge of the aerosolized liquid 102, the gas flow 150 and the liquid flow 148 may be terminated using the valves 164 and 190. In one embodiment, the valve 164 may be adjusted to a setting that vents the flow pathway 154, through which the gas flow 150 travels, such as by fluidically connecting or venting the flow pathway 154 to atmospheric conditions as indicated by the dashed arrow 196, for example. Similarly, the valve 190 may be adjusted to a setting that vents the flow pathway 152 through which the liquid flow 148 travels, such as by fluidically connecting the flow pathway 152 to atmospheric conditions or to a vacuum or low-pressure source 198. By connecting the flow pathway 152 to the vacuum source 198, drips of the liquid flow 148 may be prevented from being discharged from the sprayer 144.

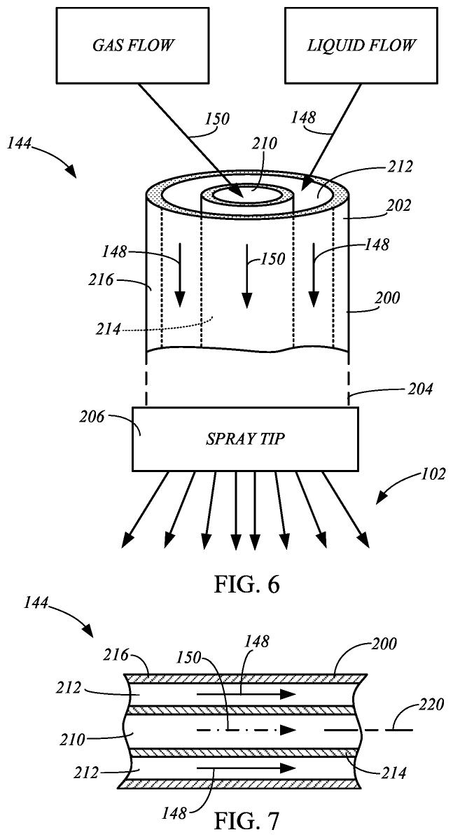

In one embodiment, the sprayer 144 comprises a nebulizing or atomizing catheter 200 having one or more lumens that receive the gas flow 150, and one or more lumens that receive the liquid flow 148, at a proximal end 202, as indicated in the simplified diagram of the sprayer 144 shown in FIG. 6. A distal end 204 of the catheter includes a spray tip 206 that combines the gas flow 150 and the liquid flow 148 and discharges the aerosolized liquid 102.

In one example, the catheter 200 includes a central lumen 210, that receives the gas flow 150, and an outer lumen 212 that receives the liquid flow 148, as indicated in FIG. 6 and the simplified cross-sectional view of the catheter provided in FIG. 7. Thus, the gas flow 150 travels through the central lumen 210 of the catheter 200, while the liquid flow 148 travels through the outer lumen 212, as shown in FIGS. 6 and 7.

The lumens 210 and 212 may respectively be formed by tubes 214 and 216. In one embodiment, the central lumen 210 and the tube 214 extend through the outer lumen 212 and the tube 216. In one example, the central lumen 210 may have a diameter of about 0.075-0.15 mm, while the outer lumen 212 may have a diameter of about 0.25-0.5 mm.

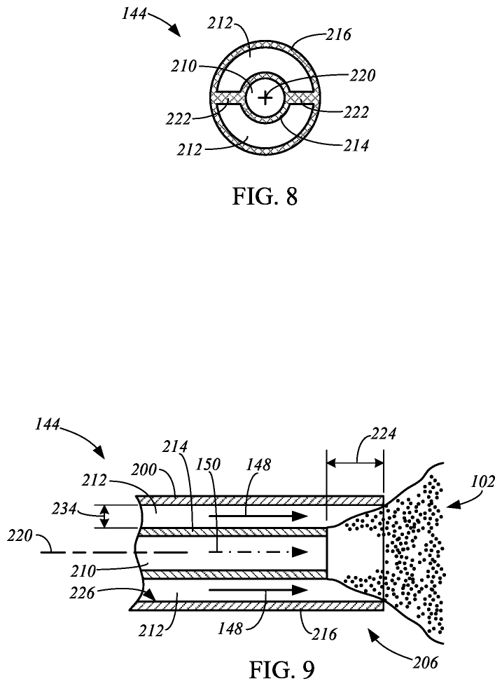

The tube 214 and the central lumen 210 may be supported in substantial alignment with a central axis 220 of the tube 216 and the lumen 212 by one or more ribs 222 such as illustrated by the cross-sectional view of the catheter 200 shown in FIG. 8. Each rib 222 extends from the inner tubing 214 or wall of the central lumen 210 to the wall of the outer tubing 216 of the catheter 200.

FIG. 9 is a simplified side cross-sectional view of an example of a spray tip 206, in accordance with embodiments of the present disclosure. In this example, the central lumen 210 at the distal end 204 is recessed along the central axis 220 relative to the outer lumen 212 by an offset distance 224. This arrangement provides enhanced the aerosolization of the liquid flow 148 as compared to a reverse arrangement where the gas flow 150 is in the outer lumen 212 and the liquid flow 148 is in the central lumen 210, and allows for low volumetric flow rates for the gas flow 150, while providing the desired aerosolization of the liquid flow 148.

For example, the gas flow 150 discharged through the central lumen 210 presses the liquid flow 148 against the inner wall 226 of the outer lumen 212, where the liquid velocity is approximately zero or near zero due to the no-slip condition boundary layer condition. Consequently, the annular liquid film on the inner wall 226 experiences strong shear forces, which break down the liquid flow 148 into fine drops. As a result, the arrangement of FIG. 9 allows for nebulization or atomization of larger volumes of liquid using smaller volumetric flow rates (e.g., 0.1-0.3 liters/minute) of the gas flow 150, that are more suitable for preterm neonates.

However, when the reverse arrangement to that illustrated in FIG. 9 is utilized, a gas-liquid interface forms at the cylindrical surface of the liquid flow discharged through the central lumen 210. Due to the lack of a no-slip boundary layer condition, the shear forces on the liquid flow from the gas flow exiting the outer lumen 212 are reduced relative to the opposite arrangement discussed above and shown in FIG. 9. As a result, there is a reduced aerosolization of the liquid flow through the central lumen 210, and higher volumetric gas flow rates (e.g., 1-10 liters/minute) are required to properly aerosolize the liquid flow, to levels that may not be suitable for preterm neonates. Accordingly, such an arrangement is less suitable for the delivery of an aerosolized surfactant to treat respiratory distress syndrome of neonates, for example.

The offset distance 224 plays an important role in the nebulization of the liquid flow 148. If the offset distance 224 is too small, the liquid film on the inner wall 226 will not thin out adequately, resulting in the undesirable formation of large drops after the liquid is initially broken up by the gas flow 150. On the other hand, if the offset distance 224 is too large, fine liquid drops coalesce within the distal tip of outer lumen 212 forming larger drops. In one example, the formation of large drops at the distal tip of the catheter 200 is reduced or prevented when the offset distance 224 is approximately 0.05-1.0 mm.

As mentioned above, the central lumen 210 may be supported within the outer lumen 212 via one or more ribs 222, such as shown in FIG. 8. Alternatively, the central lumen 210 may be disconnected from the outer lumen 212, at least at the distal end 204 of the catheter 200, resulting in the distal end of the central lumen 210 being cantilevered, such as shown in the example of FIG. 9. This allows the central lumen 210 to move radially relative to the central axis 220 and the outer lumen 212. In this configuration, the liquid flow 148 operates to maintain the central lumen 210 proximate the central axis 220 of the outer lumen 212.

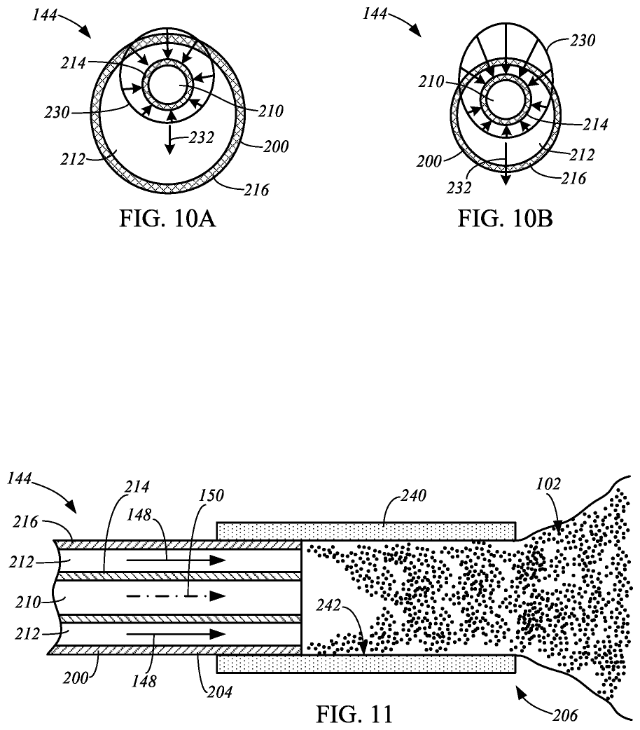

FIGS. 10A and 10B illustrate examples of pressure forces, which are represented by the arrows within the oval 230, that are applied to the central lumen 210 or tubing 214 during operation. Whenever the central lumen 210 or tubing 214 moves off-center (as shown), a net pressure force represented by arrow 232 develops and pushes the tubing 214 toward the central axis 220 of the outer lumen 212. This net force 232 is preferably large enough to overcome any resistance to the movement of the central lumen 210 and the tubing 214, thereby maintaining the lumen 210 and the tube 214, or at least their distal ends, substantially aligned with the central axis 220 of the outer lumen 212, thereby promoting the aerosolization of the liquid flow 148.

The narrower the annular gap 234 (FIG. 9) between the central lumen 210 and tubing 214 and the outer lumen 212 and tubing 216, the greater the net force 232 directing the central lumen 210 and tubing 214 back toward the central axis 220 when it moves off axis. Thus, the narrower annular gap 234 of FIG. 10B is capable of producing a greater net force against the central lumen 210 and tubing 214 when it becomes displaced from the central axis 220 of the outer lumen 212, than that produced by the example of FIG. 10A having a larger annular gap 234. Consequently, a narrow annular gap 234 between the central lumen 210 and the outer lumen 212 may be preferred to generate an adequate pressure force to maintain centration of the central lumen 210 and tube 214 during operation of the device 108. In one example, the annular gap 234 is approximately 0.015 mm to 0.035 mm.

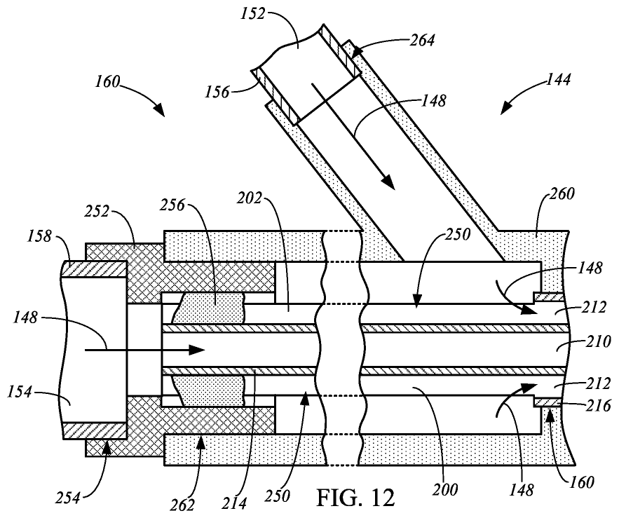

FIG. 11 illustrates another example of the spray tip 206, in accordance with embodiments of the present disclosure. In this example, the spray tip 206 comprises a sleeve 240 that is secured to the distal end 204 of the catheter 200. The sleeve 240 operates similarly to the offsetting of the central lumen 210 from the outer lumen 212 discussed above with reference to FIG. 9 by allowing the gas flow 150 to shear the fluid flow 148 along the inner wall 242 of the sleeve 240 and generate the aerosolized liquid 102.

As mentioned above, the aerosolization device 108 may include an adapter 160 (FIG. 3) for respectively delivering the gas flow 150 and the liquid flow 148 to the central lumen 210 and the outer lumen 212 of the catheter 200, as indicated in FIG. 6. One example of such an adapter 160 is provided in the side cross-sectional view of the proximal end 202 of the catheter 200 provided in FIG. 12.

In the example adapter 160, at least one opening 250 is formed in the outer tubing 216 of the proximal end 202 of the catheter 200 to provide access to the outer lumen 212. The opening 250 may extend about 1.0 cm, for example, from the proximal end 202, and may be formed by shaving the outer tubing 216. A fitting 252, such as a female luer-to-barbed fitting, may be secured to the proximal end 202 of the outer tubing. A port 254 of the fitting 252 may be connected to the tubing 158 forming the fluid flow pathway 154 through which the gas flow 150 is received. The outer lumen 212 at the proximal end 202 of the catheter 200 may be blocked using an epoxy 256 or through another suitable sealing technique. This prevents the gas flow 150 received from the tubing 158 from entering the lumen 212 through the port 254.

A fitting 260, such as a Y-fitting or another suitable arrangement, may be used to receive the proximal end 202 of the catheter 200 with the fitting 252, such as at a port 262. A side port 264 may be connected to the tubing 156 forming the flow pathway 152, through which the liquid flow 148 is received. Seals between the port 262 and the fitting 252, between the side port 264 and the tubing 156, and between an exit port 266 and the catheter 200, may be formed using an epoxy or through another suitable technique to force the liquid flow 148 into the outer lumen 212. Thus, the liquid flow 148 entering through the side port 264 of the fitting 260 is directed through the one or more openings 250 in the tubing 216 of the catheter 200 and into the outer lumen 212, as indicated in FIG. 12. Other types of adapters 160 may also be used.

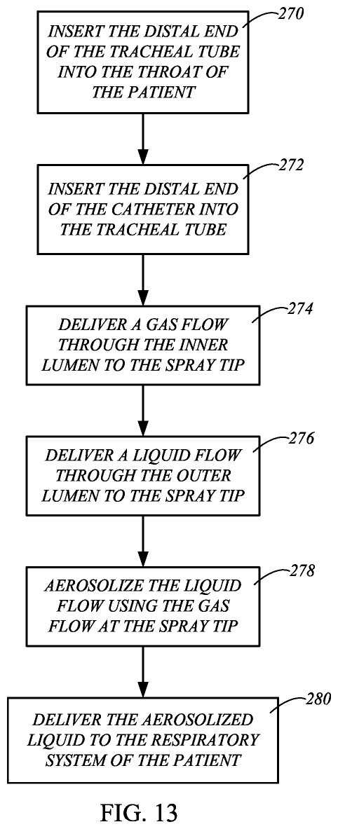

FIG. 13 is a flowchart illustrating a method for administering an aerosolized liquid to the respiratory system of a patient, in accordance with embodiments of the present disclosure. The method is performed using one or more embodiments of the system 100, which includes the patient interface 106 and the aerosolization device 108 formed in accordance with one or more embodiments described herein.

At 270 of the method, a distal end of a tracheal tube 214 of an LMA 112 is inserted into the throat 122 of the patient, such that a laryngeal cuff 120 of the LMA 112 is positioned in the pharynx 128 of the throat 122, such as shown in FIG. 2. At 272 of the method, the distal end 204 of the catheter 200 is inserted into the tracheal tube 114, and may be extended to a desired location within the tracheal tube 114, such as proximate to the distal end of the tracheal tube 114, for example.

At 274 of the method, a gas flow 150 generated by a gas flow generator 142 is delivered through an inner lumen 210 and tube 214 of the catheter 200 from a proximal end 202 to a spray tip 206, and a flow 148 of a liquid 146 discharged from a liquid delivery device 140 is delivered through an outer lumen 212 from the proximal end 202 to the spray tip 206. At 276, the liquid flow 148 is aerosolized using the gas flow 150 at the spray tip 206, and the aerosolized liquid 102 is delivered to the respiratory system 104 of the patient, such as through the tracheal tube 114.

These and other method steps may be performed in accordance with the embodiments of the system 100 described herein.

Although the embodiments of the present disclosure have been described with reference to preferred embodiments, workers skilled in the art will recognize that changes may be made in form and detail without departing from the spirit and scope of the present disclosure.

1. A system for administering an aerosolized liquid to a respiratory system of a patient, the system comprising:
a patient interface including a laryngeal mask airway comprising a tracheal tube and a laryngeal cuff located at a distal end of the tracheal tube;
an aerosolization device comprising:
a liquid delivery device configured to deliver a liquid;
a gas flow generator configured to generate a gas flow; and
a sprayer comprising a catheter configured to extend into the tracheal tube, the catheter having proximal and distal ends and including a first lumen configured to receive the gas flow at the proximal end, a second lumen configured to receive a flow of the liquid at the proximal end, and a spray tip at the distal end configured to aerosolize the flow of the liquid using the gas flow,
wherein the first lumen extends through the second lumen.

2. The system of claim 1, wherein:
the catheter includes a first tube forming the first lumen, and a second tube forming the second lumen; and
the first tube extends through the second tube.

3. The system of claim 2, wherein the catheter includes at least one rib extending between the first and second tubes, and along a longitudinal axis of the tubes.

4. The system of claim 2, wherein a distal end of the first tube is disconnected from a distal end of the second tube, wherein the distal end of the first tube is cantilevered.

5. The system of claim 2, wherein the spray tip comprises:
a distal end of the first tube and a distal end of the second tube, and the distal end of the first tube is recessed into the distal end of the second tube; or
wherein the spray tip comprises a sleeve attached to a distal end of the second tube.

6. The system of claim 2, wherein the gas flow generator includes:
a source of compressed gas;
a first valve configured to control a fluidic connection between the source of compressed gas and a first flow pathway connected to the first lumen; and
a controller configured to selectively adjust the first valve to a first valve setting, in which the source of compressed gas is fluidically connected to the first flow pathway and the gas flow travels through the first flow pathway to the first lumen, and a second valve setting, in which the source of compressed gas is fluidically disconnected to the first flow pathway and the first lumen.

7. The system of claim 6, wherein:
the first valve is configured to control a fluidic connection between the first flow pathway and atmospheric pressure; and
the controller is configured to selectively adjust the first valve to a third valve setting, in which the first valve fluidically connects the first flow pathway to atmospheric pressure and the source of compressed gas is disconnected from the first flow pathway.

8. The system of claim 6, wherein:
the liquid delivery device includes:
a pump configured to deliver the liquid from a supply of the liquid; and
a second valve fluidically coupled to the source of compressed gas, the liquid delivered by the pump, and a second flow pathway fluidically connected to the second lumen; and
the controller is configured to:
control the pump to deliver the liquid; and
selectively adjust the second valve to a first valve setting, in which the liquid delivered from the pump is fluidically connected to the second flow pathway and the source of compressed gas is fluidically disconnected from the second flow pathway, and a second valve setting, in which the source of compressed gas is fluidically connected to the second flow pathway and the liquid delivered from the pump is fluidically disconnected from the second flow pathway.

9. The system of claim 8, wherein the controller is configured to selectively adjust the second valve to a third valve setting, in which the second valve fluidically connects the second flow pathway to atmospheric pressure or a vacuum source.

10. The system of claim 8, wherein the pump is selected from the group consisting of a syringe, a syringe pump, a piston pump, a metering pump, a pneumatic pump, a gear pump, and a rotary pump.

11. The system of claim 2, wherein the liquid delivery device comprises a pump selected from the group consisting of a syringe pump, a piston pump, a metering pump, a pneumatic pump, a gear pump, and a rotary pump.

12. A method for administering an aerosolized liquid to the respiratory system of a patient using a system, which comprises:
a patient interface including a laryngeal mask airway comprising a tracheal tube and a laryngeal cuff located at a distal end of the tracheal tube; and
an aerosolization device comprising:
a liquid delivery device;
a gas flow generator; and
a sprayer comprising a catheter including first and second lumens extending from a proximal end to a distal end, and a spray tip at the distal end, wherein the first lumen extends through the second lumen,
the method comprising:
inserting the distal end of the tracheal tube into the throat of the patient and positioning the laryngeal cuff in the pharynx of the throat;
inserting the distal end of the catheter into the tracheal tube;
delivering a gas flow generated by the gas flow generator through the first lumen from the proximal end of the catheter to the spray tip;
delivering a flow of a liquid discharged from the liquid delivery device through the second lumen from the proximal end of the catheter to the spray tip;
aerosolizing the liquid using the gas flow at the spray tip; and
delivering the aerosolized liquid to the respiratory system of the patient through the tracheal tube.

13. The method of claim 12, wherein:
the catheter includes a first tube forming the first lumen, and a second tube forming the second lumen; and
the first tube extends through the second tube.

14. The method of claim 13, wherein:
the catheter includes at least one rib extending between the first and second tubes, and along a longitudinal axis of the tubes; or
a distal end of the first tube corresponding at the distal end of the catheter is disconnected from a distal end of second tube at the distal end of the catheter, wherein the distal end of the first tube is cantilevered.

15. The method of claim 13, wherein the spray tip comprises:
a distal end of the first tube and a distal end of the second tube, and the distal end of the first tube is recessed into the distal end of the second tube; or
wherein the spray tip comprises a sleeve attached to a distal end of the second tube.

16. The method of claim 13, wherein:
the gas flow generator includes:
a source of compressed gas;
a first valve configured to control a fluidic connection between the source of compressed gas and a first flow pathway connected to the first lumen; and
a controller configured to selectively adjust the first valve to a first valve setting, in which the source of compressed gas is fluidically connected to the first flow pathway and the gas flow travels through the first flow pathway to the first lumen, and a second valve setting, in which the source of compressed gas is fluidically disconnected to the first flow pathway and the first lumen; and
delivering a gas flow comprises adjusting the first valve from the second valve setting to the first valve setting using the controller.

17. The method of claim 16, including terminating delivering the aerosolized liquid to the respiratory system comprising adjusting the first valve to the second valve setting using the controller, or adjusting the valve to a third valve setting using the controller, in which the first valve fluidically connects the first flow pathway to atmospheric pressure and the source of compressed gas is fluidically disconnected from the first flow pathway.

18. The method of claim 16, wherein:
the liquid delivery device includes:
a pump configured to deliver the liquid from a supply of the liquid; and
a second valve fluidically coupled to the source of compressed gas, the liquid delivered by the pump, and a second flow pathway fluidically connected to the second lumen; and
delivering a flow of the liquid comprises:
adjusting the second valve from a second valve setting to a first valve setting, in which the second flow pathway is fluidically connected to the pump and fluidically disconnected from the source of compressed gas using the controller;
driving the pump to discharge the liquid from the supply;
delivering the discharged liquid to the second flow pathway through the second valve;
adjusting the second valve to a second valve setting, in which second flow pathway is fluidically connected to the source of compressed gas and fluidically disconnected from the pump;
delivering a second gas flow from the source of compressed gas through the second valve and into the second flow pathway; and
delivering the flow of the liquid discharged from the pump through the second lumen in response to delivering the second gas flow.

19. The method of claim 18, wherein the method includes terminating delivering the flow of the liquid comprising:
terminating driving the pump using the controller; and
adjusting the valve to a third valve setting, in which the second flow pathway is fluidically connected to atmospheric pressure or a vacuum source, and is fluidically disconnected from the pump and the source of compressed gas.

20. The method of claim 18, wherein the pump is selected from the group consisting of a syringe, a syringe pump, a piston pump, a metering pump, a pneumatic pump, a gear pump, and a rotary pump.
